(19) United States
(12) Patent Application Publication
(10) Pub. No.: US 2006/0217791 A1
(43) Pub. Date: Sep. 28, 2006

(54) Multi-lumen catheter having external electrical leads

(75) Inventors: Mark J. Spinka, Reading, PA (US); Mark T. Salzberger, Sinking Spring, PA (US)

Correspondence Address: AMSTER, ROTHSTEIN & EBENSTEIN LLP, 90 PARK AVENUE, NEW YORK, NY, 10016, US

(73) Assignee: ARROW INTERNATIONAL, INC.

(21) Appl. No.: 11/091,786

(22) Filed: Mar. 23, 2005

(51) Int. Cl. A61N 1/05

(52) U.S. Cl. 607/116

(57) ABSTRACT

A catheter including a lead body and at least one lumen extending longitudinally through the lead body. At least one conductor is formed on an outside surface of the lead body and extends from a proximal end portion of the lead body to a distal end portion of the lead body.

### TECHNICAL FIELD

The present invention is related to catheters having electrical leads.

### BACKGROUND

In order for a catheter to deliver and/or receive electrical signals from a patient's body, it is necessary to provide a structure for carrying an electrical impulse between the proximal end of the catheter to the distal end. This is conventionally accomplished by either providing one or more electrical wires in a lumen of the catheter or by embedding a wire in the wall of the catheter. For example, U.S. Pat. No. 5,584,873 (“the '873 Patent”) discloses a medical electrical lead including an elongated lead body provided with multiple conductor lumens each containing a conductor and at least one compression lumen which does not contain a conductor.

Conventional catheters which include electrical leads are susceptible to problems such as kinking. During kinking, the lumens lying within a kinking plane may collapse, thereby preventing flow of fluid through the lumens. Prior art catheters have attempted to diminish the effect of kinking by arranging the lumens such that not all the lumens lay in the same kinking plane. Thus, at least one of the lumens would remain open during kinking. This may assist in preserving a catheter's ability to deliver fluid, but during kinking a further problem may develop in that the electrical leads within the catheter may become damaged, thereby deteriorating or in some cases eliminating the catheters ability to deliver and/or receive electrical impulses. Further, forming catheters with internal electrical leads requires a somewhat complicated and expensive manufacturing process.

Accordingly, there is a need for a catheter having electrical leads that are not susceptible to damage when the catheter is kinked and/or bent and that can be manufactured easily and inexpensively.

### SUMMARY OF THE INVENTION

One aspect of the invention provides a catheter that is able to deliver and/or receive electrical impulses reliably even when subjected to kinking and/or bending.

Another aspect of the invention provides a catheter that is relatively kink resistant.

Another aspect of the invention provides a catheter having electrical conductors which are less likely to be damaged when the catheter is bent and/or kinked.

A catheter according to an exemplary embodiment of the invention includes a lead body, at least one lumen extending longitudinally through the lead body, and at least one conductor formed on an outside surface of the lead body and extending from a proximal end portion of the lead body to a distal end portion of the lead body.

A method of forming a catheter according to an exemplary embodiment of the invention includes forming a lead body comprising at least one lumen extending longitudinally through the lead body, and disposing at least one conductor on an outside surface of the lead body, the at least one conductor extending from a proximal end portion of the catheter to a distal end portion of the catheter.

In at least one embodiment, the at least one conductor is a wire.

In at least one embodiment, the at least one conductor extends helically from the proximal end portion to the distal end portion of the lead body.

In at least one embodiment, multiple conductors are formed on the outside surface of the lead body.

In at least one embodiment, an electrically insulating layer is formed over at least a portion of the at least one conductor.

In at least one embodiment, the electrically insulating layer is not formed over the at least one conductor at at least one of the proximal end portion and the distal end portion of the lead body.

In at least one embodiment, an electrode is formed at at least one of the proximal end portion and the distal end portion of the lead body.

In at least one embodiment, a conductive band is formed around at least one of the proximal end portion and the distal end portion of the lead body.

In at least one embodiment, at least a portion of the insulating layer is stiffer than at least one other portion of the insulating layer.

In at least one embodiment, the at least one lumen has a rounded cross-sectional shape.

In at least one embodiment, multiple lumens extend longitudinally through the lead body.

In at least one embodiment, three lumens extend longitudinally through the lead body.

In at least one embodiment, at least one other conductor extends through the at least one lumen.

In at least one embodiment, at least one other conductor extends through the lead body.

These and other features of this invention are described in, or are apparent from, the following detailed description of various exemplary embodiments of this invention.

### BRIEF DESCRIPTION OF THE DRAWINGS

Various exemplary embodiments of this invention will be described in detail, with reference to the following figures, wherein:

FIG. 1 is a perspective view of a catheter according to an exemplary embodiment of the invention; and

FIG. 2 is a perspective view of a catheter according to another exemplary embodiment of the invention.

### DETAILED DESCRIPTION OF PREFERRED EMBODIMENTS

The various exemplary embodiments of the present invention are directed to a catheter having conductors formed around the outside of the catheter body. Forming the conductors external to the catheter body diminishes the risk that the conductors will be damaged during bending and/or kinking of the catheter. Further, by arranging the conductors in specific configurations, such as, for example, a helical configuration, risk of damage can be diminished even further.

In the following detailed description, the terms “distal” and “proximal” will be used. As used herein, the term “proximal” refers to that region, portion or end of a device or procedure nearest the person using the device or performing the procedure, while the term “distal” refers to that region, portion or end of a device or procedure nearest a patient upon whom the device is being used or the procedure is being performed.

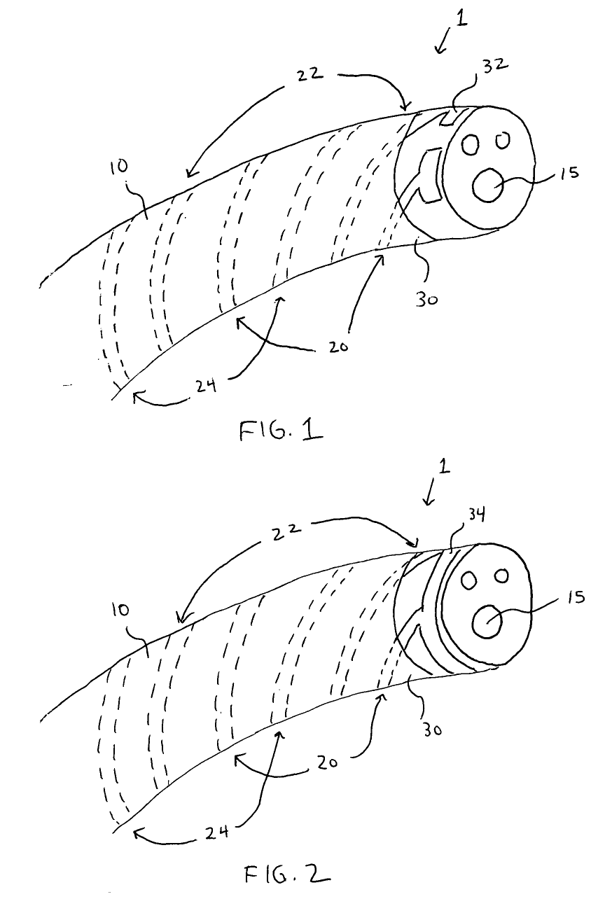

FIG. 1 is a perspective view of a catheter according to an exemplary embodiment of the invention. The catheter 1 includes a lead body 10 and a number of lumens 15 extending longitudinally through the lead body 10. Although three lumens 15 are shown in FIG. 1, any number of lumens may be formed in the lead body in any type of arrangement. However, a three-lumen configuration is preferred. Further, as shown in FIG. 1, the three lumens are preferably arranged in a triangular configuration so that not all the lumens lay in the same kinking plane and at least one of the lumens will remain open during kinking of the catheter. Although the lumens 15 preferably have rounded cross-sections so as to provide more kink resistance, the lumens 15 may have any other suitable cross-sectional shape.

The lead body 10 may be made from a variety of sterilizable plastics known in the art, including, but not limited to, polyamides and copolymers thereof, low density polyethylene, high density polyethylene, polypropylene, polystyrene, polycarbonate, polytetrafluoroethylene, fluorinated ethylene propylene and polyurethane. The lead body 10 is preferably formed by extrusion.

Flexible electrical conductors 20, 22, 24 are formed around the outer surface of the lead body 10. Although the conductors 20, 22, 24 are shown helically arranged around the lead body 10, the conductors 20, 22, 24 may be configured in any way around the lead body 10, such as in a straight line or arranged in a circuit pattern. However, the conductors 20, 22, 24 are preferably helically arranged in a “barber-pole” configuration so that they are less likely to break upon bending or stretching. Further, multiple conductors are preferred to ensure that an electrical path exists even when one of the conductors is damaged. The conductors 20, 22, 24 may be made of any suitable electrically conducting material, such as, for example, Au, Ag, Pt, Ni and Cu. Further, although three conductors are shown in FIG. 1, any number of conductors may be formed around the lead body 10.

The conductors 20, 22, 24 may be formed directly on the lead body by any suitable method, such as, for example, chemical vapor deposition, laser deposition, and photolithography techniques involving masking and spraying, brushing or dipping operations. Alternatively, each of the conductors 20, 22, 24 may be in the form of separate wires that are attached to the outer surface of the lead body 10 by a suitable adhesive.

The catheter 1 further includes an insulating layer 30 formed over the conductors 20, 22, 24. As shown in FIG. 1, the insulating layer 30 preferably covers the outer surface of the lead body 10 except for proximal and distal end portions of the lead body 10. With this configuration, the ends of the conductors 20, 22, 24 are exposed for electrical contact with a medical device and/or a nerve ending, for instance. Alternatively, the insulating layer 30 may be formed only over the conductors 20, 22, 24, so that the insulating layer 30 follows the same path as the conductors 20, 22, 24. The insulating layer 30 may be made of any suitable biocompatible and electrically insulating material, such as, for example, silicon rubber, polystyrene, polyurethane, parylene, polyethylene, polyvinyl chloride, polyolefin, polyester and other thermoplastics. The insulating material may be deposited by using, for example, chemical vapor deposition, laser deposition, and/or photolithography techniques.

In conventional catheters, a stylet wire is typically inserted into one of the lumens to stiffen and manipulate the catheter during implantation. In the present embodiment, the stiffness of the insulating layer 30 may be “tuned” so as to obviate the need for a stylet, such as by altering the catheter wall thickness and/or the catheter material along the length of the catheter. For example, the insulating layer 30 may be made stiffer at portions of the lead body 10 such as with a dual-durometer extrusion to diminish the risk of kinking and bending during insertion of the lead body 10 and to aid in manipulation of the lead body 10.

The conductors 20, 22, 24 may be attached to electrodes 32 formed at the exposed proximal and distal end portions of the lead body 10. Alternatively, as shown in FIG. 2, conductive band 34 may be formed around the proximal and distal end portions of the lead body 10. The conductive bands 34 may be used to provide a uniform signal.

While particular embodiments of the present invention have been illustrated and described, it would be obvious to those skilled in the art that various other changes and modifications can be made without departing from the spirit and scope of the invention. For example, in other embodiments of the invention, conductors may be disposed within at least one of the lumens in addition to the conductors disposed on the outside of the lead body. It is therefore intended to cover in the appended claims all such changes and modifications that are within the scope of this invention.

What is claimed is:

1. A catheter comprising:
a lead body;
at least one lumen extending longitudinally through the lead body; and
at least one conductor formed on an outside surface of the lead body and extending from a proximal end portion of the lead body to a distal end portion of the lead body.

2. The catheter of claim 1, wherein the at least one conductor is a wire.

3. The catheter of claim 1, wherein the at least one conductor extends helically from the proximal end portion to the distal end portion of the lead body.

4. The catheter of claim 1, wherein multiple conductors are formed on the outside surface of the lead body.

5. The catheter of claim 1, wherein a single conductor is formed on the outside surface of the lead body.

6. The catheter of claim 1, further comprising an electrically insulating layer formed over at least a portion of the at least one conductor.

7. The catheter of claim 6, wherein the electrically insulating layer is not formed over the at least one conductor at at least one of the proximal end portion and the distal end portion of the lead body.

8. The catheter of claim 7, further comprising an electrode formed at at least one of the proximal end portion and the distal end portion of the lead body.

9. The catheter of claim 7, further comprising a conductive band formed around at least one of the proximal end portion and the distal end portion of the lead body.

10. The catheter of claim 1, wherein at least a portion of the insulating layer is stiffer than at least one other portion of the insulating layer.

11. The catheter of claim 1, wherein the at least one lumen has a rounded cross-sectional shape.

12. The catheter of claim 1, wherein multiple lumens extend longitudinally through the lead body.

13. The catheter of claim 12, wherein three lumens extend longitudinally through the lead body.

14. The catheter of claim 1, further comprising at least one other conductor extending through the at least one lumen.

15. The catheter of claim 1, further comprising at least one other conductor extending through the lead body.

16. A method of forming a catheter, comprising:
forming a lead body comprising at least one lumen extending longitudinally through the lead body; and
disposing at least one conductor on an outside surface of the lead body, the at least one conductor extending from a proximal end portion of the catheter to a distal end portion of the catheter.

17. The method of claim 16, wherein the at least one conductor is a wire and the step of disposing comprises adhering the at least one conductor to the outside surface of the lead body.

18. The method of claim 16, wherein the step of disposing comprises at least one of: chemical vapor deposition, laser deposition and photolithography.

19. The method of claim 16, wherein the step of forming a lead body comprises extrusion.

20. The method of claim 16, wherein the at least one conductor is disposed so as to extend helically from the proximal end portion to the distal end portion of the lead body.

21. The method of claim 16, wherein a single conductor is formed on the outside surface of the lead body.

22. The method of claim 16, wherein multiple conductors are formed on the outside surface of the lead body.

23. The method of claim 16, further comprising forming an electrically insulating layer over at least a portion of the at least one conductor.

24. The method of claim 23, wherein the electrically insulating layer is not formed over the electrically insulating layer at at least one of the proximal end portion and the distal end portion of the lead body.

25. The method of claim 24, further comprising forming an electrode at at least one of the proximal end portion and the distal end portion of the lead body.

26. The method of claim 24, further comprising forming a conductive band around at least one of the proximal end portion and the distal end portion of the lead body.

27. The method of claim 16, wherein at least a portion of the insulating layer is stiffer than at least one other portion of the insulating layer.

28. The method of claim 16, wherein the at least one lumen has a rounded cross-sectional shape.

29. The method of claim 16, wherein the lead body comprises multiple lumens extending longitudinally through the lead body.

30. The method of claim 29, wherein the lead body comprises three lumens extending longitudinally through the lead body.
